(19) United States
(12) Patent Application Publication
(10) Pub. No.: US 2024/0416052 A1
(43) Pub. Date: Dec. 19, 2024

(54) BREATHING SIMULATION SYSTEM

(71) Applicant: HCMed Innovations Co., LTD., Taipei City (TW)

(72) Inventors: CHUN-CHIA JUAN, New Taipei City (TW); CHANG-HSIEH YAO, Taipei City (TW); HSIN-YI PAI, New Taipei City (TW)

(21) Appl. No.: 18/488,956

(22) Filed: Oct. 17, 2023

(30) Foreign Application Priority Data
Jun. 14, 2023 (CN) 202310703106.7

(51) Int. Cl. A61M 11/00; A61M 16/00

(52) U.S. Cl. CPC A61M 11/00; A61M 16/0003; A61M 2205/07; A61M 2205/3358; A61M 2205/70

(57) ABSTRACT

A breathing simulation system is used to test a plurality of to-be-tested atomization devices and includes a main tube, a plurality of connecting pipes, an air suction mechanism, a liquid supply mechanism and an aerosol condensation module. The main tube forms a main airflow passage, and is provided with air intake holes and at least one liquid outlet. Each of the connecting pipes is disposed between the corresponding air intake hole and an air suction port of the corresponding to-be-tested atomization device. The air suction mechanism communicates with the main airflow passage, and is configured to generate a negative pressure in the main airflow passage, and form an airflow path between the air suction mechanism and each of the air suction ports.

### CROSS-REFERENCE TO RELATED PATENT APPLICATION

This application claims the benefit of priority to Patent Application No. 202310703106.7, filed on Jun. 14, 2023, in the People's Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a simulation system, and more particularly to a breathing simulation system capable of testing atomization devices in large quantities.

### BACKGROUND OF THE DISCLOSURE

In order to test whether or not an atomization device can pass a reliability test, it is necessary to repeatedly provide medicinal liquid to the atomization device over a long period of time, and connect the atomization device to an air pump, so as to simulate human inhalation. However, such a testing method lacks efficiency and a mechanism that can effectively collect the aerosol generated by the atomization device, so that the scattered aerosol causes pollution.

More specifically, there is no dedicated equipment in the relevant art that can test a large quantity of atomization devices while automatically replenishing medicinal liquid and collecting the waste aerosol, which contributes to an inconvenient experience in the testing process of the atomization devices.

### SUMMARY OF THE DISCLOSURE

In response to the above-referenced technical inadequacies, the present disclosure provides a breathing simulation system capable of testing a large quantity of atomization devices.

In one aspect, the present disclosure provides a breathing simulation system for testing a plurality of to-be-tested atomization devices. The breathing simulation system includes a main tube, a plurality of connecting pipes, an air suction mechanism, a liquid supply mechanism and an aerosol condensation module. The main tube forms a main airflow passage, and is provided with air intake holes and at least one liquid outlet thereon. Each of the connecting pipes is disposed between the corresponding air intake hole and an air suction port of the corresponding to-be-tested atomization device. The air suction mechanism communicates with the main airflow passage, and is configured to generate a negative pressure in the main airflow passage, and form an airflow path between the air suction mechanism and each of the air suction ports.

In another aspect, the present disclosure provides a breathing simulation system used to test a to-be-tested atomization device and includes a main tube, a connecting pipe, an air suction mechanism, a liquid supply mechanism and an aerosol condensation module. The main tube forms a main airflow passage, and is provided with an air intake hole and at least one liquid outlet thereon. The connecting pipe is disposed between the air intake hole and an air suction port of the to-be-tested atomization device. The air suction mechanism communicates with the main airflow passage, and is configured to generate a negative pressure in the main airflow passage, and form an airflow path between the air suction mechanism and the air suction port.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a simplified schematic diagram of a breathing simulation system according to one embodiment of the present disclosure;

FIG. 2 is a schematic diagram of a to-be-tested atomization device according to one embodiment of the present disclosure;

FIG. 3 is a schematic diagram of a drug administration module of the to-be-tested atomization device;

FIG. 4 is a flowchart of a test process in one embodiment of the present disclosure; and

FIG. 5 is a schematic diagram showing details of the breathing simulation system according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

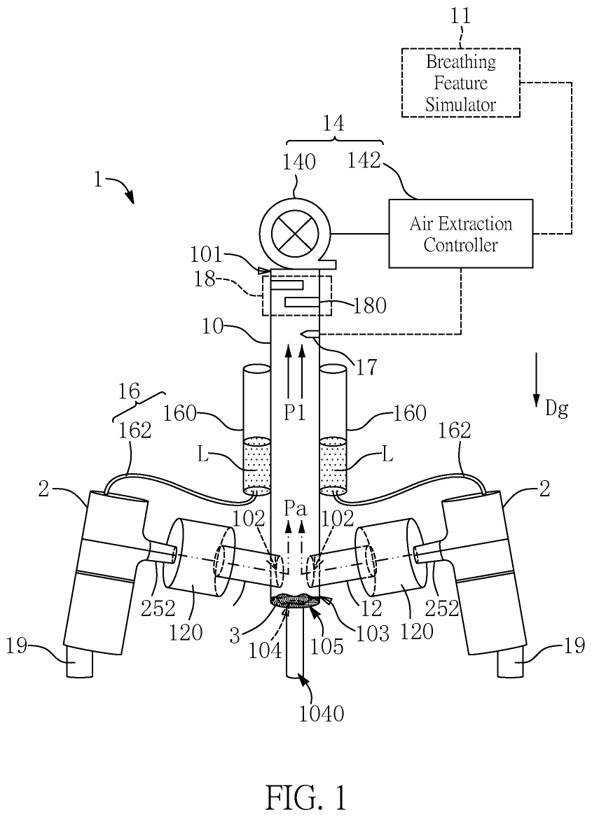

FIG. 1 is a simplified schematic diagram of a breathing simulation system according to one embodiment of the present disclosure. Reference is made to FIG. 1, in which one embodiment of the present disclosure provides a breathing simulation system 1 for testing a plurality of to-be-tested atomization devices 2. The breathing simulation system 1 includes a main tube 10, a plurality of connecting pipes 12, an air suction mechanism 14, a liquid supply mechanism 16 and an aerosol condensation module 18.

The main tube 10 forms a main airflow passage P1. The main body 10 can be, for example, a cylindrical tube arranged along a gravity direction Dg, and a plurality of air intake holes 102 and at least one liquid outlet (e.g., a liquid discharge hole 104) are arranged on a tube wall of the main tube 10. The air intake holes 102 can be arranged around a central axis of the main tube 10, and the plurality of connecting pipes 12 are respectively connected to the corresponding air intake holes 102, respectively. In some embodiments, the main tube 10 has an upper end 101 and a lower end 103, the main tube 10 is connected to a lower plate 105 at the lower end 103, and the liquid discharge hole 104 is disposed on the lower plate 105 and penetrates therethrough.

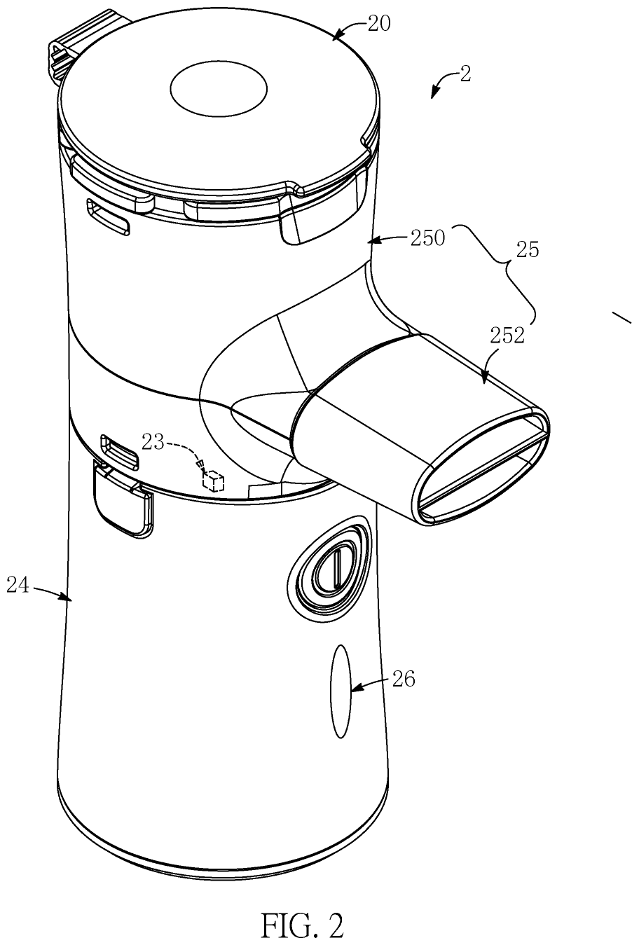

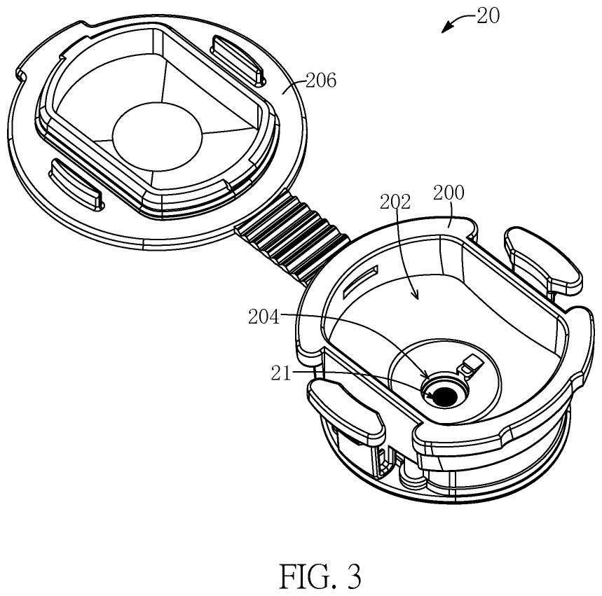

The to-be-tested atomization device 2 according to one embodiment of the present disclosure will be described with an example below. Reference is made to FIGS. 2 and 3, in which FIG. 2 is a schematic diagram of a to-be-tested atomization device according to one embodiment of the present disclosure, and FIG. 3 is a schematic diagram of a drug administration module of the to-be-tested atomization device. As shown in FIG. 2 and FIG. 3, the to-be-tested atomization device 2 can include, for example, a drug administration module 20 and an atomization module 21. The drug administration module 20 includes a housing 200, and has a liquid storage tank 202 formed in the housing 200 and a through hole 204 communicating with the liquid storage tank 202. In addition, the drug administration module 20 further includes a movable cover 206 movably disposed on the housing 200.

It should be noted that, as shown in FIG. 2, the to-be-tested atomization device 2 can include a host casing 24, an atomization kit 25 arranged on the host casing 24 and a processor provided in the host casing 24. In this way, the atomization module 21 can be powered for atomizing the medicinal liquid stored in the liquid storage tank 202.

In addition, the atomization kit 25 includes an atomization kit housing 250 disposed between the drug administration module 20 and the host housing 24, and an air suction port 252 extending from the atomization kit housing 250. A part of the air suction port 252 (for example, an upper part) is used for allowing the user to inhale an atomized aerosol after the atomization module 21 atomizes the medicinal liquid in the liquid storage tank 202, and another part (for example, a lower part) of the air suction port 252 is used for a first sensor 23 disposed in the host housing 24 to sense a pressure generated when the user inhales the atomized aerosol. In this way, when the first sensor 23 detects that a negative pressure is generated, the atomization module 21 can be driven to atomize the medicinal liquid contained in the liquid storage tank 202 to generate the atomized aerosol for the user to inhale. For example, the first sensor 23 can be a pressure sensor, which may be disposed in an accommodating space in the atomization kit 25 that communicates with a lower half of the air suction port 252.

However, the above-mentioned example is only one possible embodiment of the to-be-tested atomization device 2, and the present disclosure is not limited thereto. The to-be-tested atomization device 2 can only include a basic structure constructed by the atomization module 21 and a housing formed with the liquid storage tank 202 and the air suction port 252.

Further referring to FIG. 1, a plurality of to-be-tested atomization devices 2 can be arranged around the main tube 10 corresponding to a plurality of connecting pipes 12, such that each of the connecting pipes 12 is located between the corresponding air intake hole 102 and the air suction port 252 of the corresponding to-be-tested atomization device 2. In some embodiments, an airtight member 120 is provided at a connection between each of the connecting pipes 12 and the corresponding air suction port 252 for sealing the connection. The airtight member 120 can be made of an elastic material such as rubber, whereby the connecting pipe 12 and the corresponding air suction port 252 can be connected in an airtight manner, such that the negative pressure formed in the main tube 10 can further extend to the inside of the to-be-tested atomization device 2.

On the other hand, as shown in FIG. 1, the air suction mechanism 14 communicates with the main airflow passage P1, and is configured to extract air from the upper end 101 to generate negative pressure in the main airflow passage P1, and form an airflow path Pa between the air suction mechanism 14 and each of the air suction ports 252. In some embodiments, the air suction mechanism 14 includes a suction pump 140 and an air extraction controller 142. The suction pump 140 can be, for example, an axial blower, which is disposed at one end of the main tube 10. The air extraction controller 142 can be, for example, a microprocessor or a microcontroller, which is electrically connected to the suction pump 140, and is configured to turn on and off the suction pump 140 in a predetermined pattern, such that the negative pressure changes in the main airflow passage P1 according to the predetermined pattern. However, the present disclosure is not limited thereto. In the air suction mechanism 14, a combination of a solenoid valve and the suction pump 140 can be utilized, such that a mechanism of simulating inhalation can be achieved by using the air extraction controller 142 to open and close the solenoid valve in the predetermined pattern.

In one preferred embodiment, the suction pump 140 can preferably provide, for example, a negative pressure greater than 10 pa and a flow rate of 100 liters/minute to the main tube 10. In some embodiments, the breathing simulation system 1 further includes a barometer 17 disposed in the main airflow passage P1 for detecting a pressure in the main airflow passage P1. Optionally, the barometer 17 can be electrically connected to the air extraction controller 142. Therefore, in response to the air extraction controller 142 determining that the pressure of the main airflow passage P1 deviates from a predetermined pressure according to the pressure detected by the barometer 17, the air extraction controller 142 can adaptively adjust the negative pressure generated in the main airflow passage P1 to maintain the pressure at the predetermined pressure.

In FIG. 1, the liquid supply mechanism 16 includes a plurality of liquid supply containers 160 and a plurality of liquid supply tubes 162. Each of the liquid supply containers 160 can be used to accommodate a test liquid L, and is connected to the corresponding to-be-tested atomization device 2 through the corresponding liquid supply tube 162. For example, the liquid supply container 160 can be a hard tube made of transparent material, and can be connected to the liquid supply tube 162. The liquid supply tube 162 can penetrate through the movable cover 206 of the to-be-tested atomization device 2 and extend into the liquid storage tank 202. In this way, the liquid supply container 160 can be connected to the corresponding liquid storage container 202 of the to-be-tested atomization device 2 through the liquid supply tube 162, such that the test liquid L in the liquid supply container 160 can be provided to the corresponding liquid storage tank 202 through the liquid supply tube 162.

Therefore, under the above architecture, a test process can be performed for the to-be-tested atomization device 2.

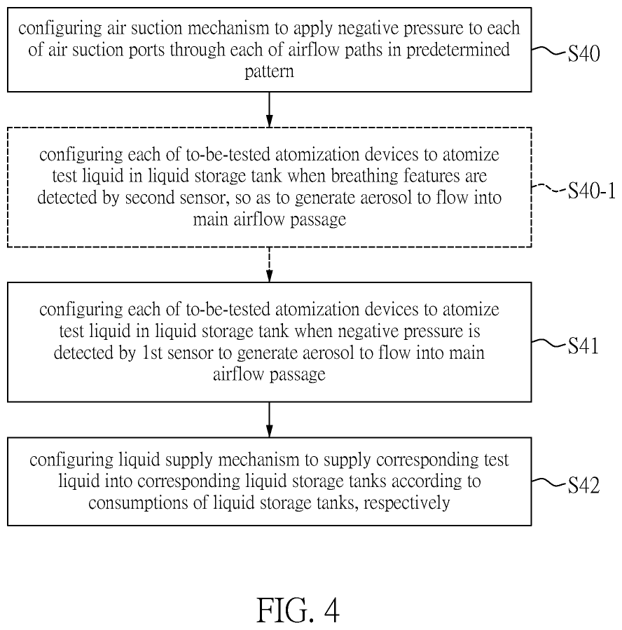

Reference is made to FIG. 4, which is a flowchart of a test process in one embodiment of the present disclosure. As shown in FIG. 4, the test process can include the following steps:

Step S40: configuring the air suction mechanism to apply the negative pressure to each of the air suction ports through each of the airflow paths in the predetermined pattern.

Step S41: configuring each of the to-be-tested atomization devices to atomize the test liquid in the liquid storage tank when the negative pressure is detected by a first sensor to generate an aerosol to flow into the main airflow passage.

Step S42: configuring the liquid supply mechanism to supply the corresponding test liquid into the corresponding liquid storage tanks according to consumptions of the liquid storage tanks, respectively.

In detail, the air extraction controller 142 can be configured to control the suction pump 140 to apply a negative pressure to each of the air suction ports 252 through the airflow path Pa in the predetermined pattern. The predetermined pattern can mimic a frequency of human inhalation, for example, inhaling for two seconds and then pausing for two seconds, which is taken as a cycle to be repeated. It should be noted that although the above test process only simulates the behavior of inhalation, the present disclosure is not limited thereto. For example, in other embodiments, an inflation mechanism including an air pump can also be provided, the inflation mechanism can be controlled by a controller to apply a positive pressure in the main tube 10, so as to simulate an exhalation when the user uses the to-be-tested atomization device 2.

As the test liquid in the liquid storage tank 202 decreases, the test liquid L in the liquid supply container 160 can be replenished in a fixed amount to the liquid tank 202 of the to-be-tested atomization device 2, such that the test liquid in the liquid storage tank 202 can be maintained at a certain height, thereby achieving a mechanism of automatic replenishment of the medicinal liquid by a fixed dosage. In one embodiment, the above-mentioned mechanism of automatically quantitatively replenishing the medicinal liquid can be realized by providing a peristaltic pump on the liquid supply tube 162, or by a common drip flow regulator, and the present disclosure is not limited thereto. In this way, such mechanism adopted in the present disclosure can achieve automatic dosage delivery without complicated design.

Further referring to FIG. 1, the aerosol condensation module 18 can be disposed in the main tube 10, and is used to condense at least a part of the aerosol generated when the to-be-tested atomization device 2 performs the aforementioned test process into waste liquid, which is then discharged through the liquid discharge hole 104. In some embodiments, the aerosol condensation module 18 includes one or more baffles 180 disposed near the upper end 101. When the negative pressure is generated, the atomized aerosol generated by the to-be-tested atomization device 2 will flow along the airflow channel Pa and the main airflow passage P1 and flow out towards the upper end 101. Therefore, the one or more baffles 180 can be arranged on this airflow path to block at least a part of the aerosol by hindering the flow of the aerosol, such that the waste liquid 3 condenses on the baffles 180. When the waste liquid 3 flows downward by the effect of gravity, it can be discharged from the liquid discharge hole 104 located below. In some embodiments, a liquid discharge pipe 1040 can be further provided to guide the waste liquid 3 to a waste liquid container for collection/storage.

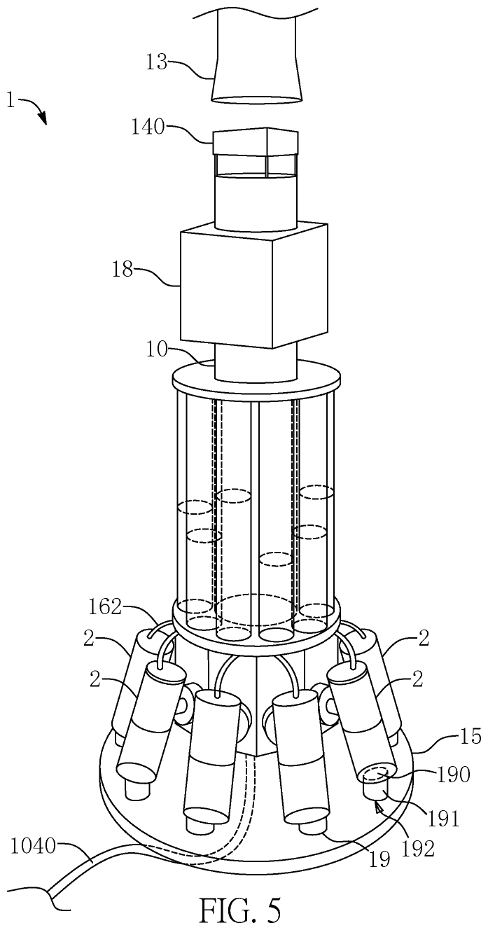

Reference is made to FIG. 5, which is a schematic diagram showing details of the breathing simulation system according to one embodiment of the present disclosure. As shown in FIG. 5, by arranging multiple ones of the to-be-tested atomization device 2 around the main tube 10, the breathing simulation system 1 provided by the present disclosure can simultaneously test a large quantity of the to-be-tested atomization devices 2 while automatically replenishing medicinal liquid and collecting the aerosol that is generated. When testing a large quantity of to-be-tested atomization devices 2 at the same time, the breathing simulation system 1 of the present disclosure can provide the same test conditions for each to-be-tested atomization device 2, such that test conditions of each air suction port 252 (such as a pumping volume or a negative pressure value) are consistent. In addition, the to-be-tested atomization device 2 is easy to be assembled, disassembled, and fixed to the breathing simulation system 1, and the breathing simulation system 1 can stably drive the multiple ones of the to-be-tested atomization device 2 for spraying the aerosol. In this embodiment, the breathing simulation system 1 can include a carrying base 15 and a plurality of positioning mechanisms 19 for carrying the to-be-tested atomization devices 2, respectively. The positioning mechanisms 19 are disposed on a periphery of the main tube 10, for example, and can be arranged around the main tube 10 to make more efficient use of space.

In detail, each of the positioning mechanisms 19 can be, for example, a cylinder having an upper surface 190, a side surface 191 and a lower surface 192. The lower surface 192 is substantially parallel to the ground and contacts the carrying base 15, while the upper surface 190 is an inclined surface inclined at a predetermined angle relative to the ground. The upper surface 190 is used to carry the corresponding to-be-tested atomization device 2. When the to-be-tested atomization device 2 is disposed on the inclined upper surface 190, the corresponding liquid container 202 will be inclined relative to the ground at the predetermined angle.

Corresponding to the above configuration, as shown in FIG. 1, each of the connecting pipe 12 is also inclined relative to the ground at the predetermined angle. In one preferred embodiment, the predetermined angle can be, for example, in a range from 10 degrees to 30 degrees, and can preferably be 15 degrees. Therefore, when the connecting pipe 12 is similarly inclined relative to the ground at the predetermined angle, the condensed waste liquid formed when the medicine aerosol contacts the air suction port 252 and pipe wall of the connecting pipe 12 will flow into the main tube 10 by the effect of gravity, thereby preventing the waste liquid from staying in the air suction port 252 and the connecting pipe 12, so as to provide an automatic waste liquid removal mechanism for the air suction port 252 and the connecting pipe 12.

On the other hand, since the aerosol condensation module 18 may not be able to completely condense all the aerosol generated by the to-be-tested atomization device 2, as shown in FIG. 5, the breathing simulation system 1 can further include an aerosol collecting tube 13 on an exhaust side of the suction pump 140. The aerosol collecting tube 13 can be connected to another waste liquid containing device to collect the waste liquid.

Reference is made to FIG. 1. In some embodiments, the breathing simulation system 1 further includes a breathing feature simulator 11 electrically connected to the air suction mechanism 14 (for example, connected to the air extraction controller 142). The breathing feature simulator 11 can be used to simulate one or more of a plurality of breathing features for generating corresponding negative pressure. For example, the breathing features can include a breathing sound, a breathing action, and one or more breathing signals generated by detecting breathing-related biological characteristics. The breathing signal can be, for example, a signal generated by detecting biometric characteristics through voltage (or wireless signal), and the breathing action can be, for example, a series of images showing rising and falling of the chest or similar characteristics.

More specifically, as shown in FIG. 2, in one specific embodiment, the to-be-tested atomization device 2 can be provided with a second sensor 26, which is used to detect breathing features of the user different from that detected by the first sensor, and correspondingly controls the atomization module 21 to operate when the detected breathing feature indicates that the user is inhaling. The second sensor 26 can be, for example, a sensor for sensing the aforementioned breathing features, for example, a sound sensor, an image sensor, an optical sensor or a wireless signal (such as WI-FI®, BLUETOOTH®) receiver. Therefore, the breathing feature simulator 11 can be, for example, a speaker, a display, a light emitting device, or a signal generator, corresponding to the manners by which the second sensor 26 detects breathing, so as to provide corresponding breathing features through sound, light, and electrical signals. Therefore, the to-be-tested atomization device 2 can control the atomization module 21 to conduct atomization to complete the test process for the to-be-tested atomization device 2.

Therefore, as shown in FIG. 4, the test process can optionally include step S40-1: configuring each of the to-be-tested atomization devices to atomize the test liquid in the liquid storage tank when one or more of the breathing features are detected by a second sensor, so as to generate the aerosol to flow into the main airflow passage.

It should be noted that, in the above-mentioned embodiments, the breathing simulation system 1 provided by the present disclosure is provided with the connecting pipes 12 and the liquid supply mechanism 16 on a premise that multiple ones of the to-be-tested atomization devices 2 are simultaneously tested. However, the present disclosure is not limited thereto. In one specific embodiment, the breathing simulation system 1 can only be provided with one connection tube 12, one liquid supply container 160, one liquid supply tube 162 and one positioning mechanism 19, so as to be suitable for testing a single to-be-tested atomization device 2.

### Beneficial Effects of the Embodiments

In conclusion, in the breathing simulation system provided by the present disclosure, a large quantity of to-be-tested atomization devices can be simultaneously tested while automatically replenishing medicinal liquid and collecting the aerosol generated.

In addition, the breathing simulation system provided by the present disclosure can achieve the effect of automatic dosage delivery without needing to design different dosage delivery conditions for multiple atomization devices to be tested.

Furthermore, through the design of the positioning mechanism, the air suction ports and the connecting pipes for the to-be-tested atomization devices can be inclined relative to the ground at the predetermined angle, which can prevent the waste liquid from accumulating in the air suction ports and the connecting pipes, so as to provide the automatic waste liquid removal mechanism.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A breathing simulation system for testing a plurality of to-be-tested atomization devices, and the breathing simulation system comprising:
a main tube forming a main airflow passage, wherein the main tube is provided with a plurality of air intake holes and at least one liquid outlet;
a plurality of connecting pipes, each disposed between the corresponding air intake hole and an air suction port of the corresponding to-be-tested atomization device; and
an air suction mechanism communicating with the main airflow passage, wherein the air suction mechanism is configured to generate a negative pressure in the main airflow passage, and form an airflow path between the air suction mechanism and each of the air suction ports.

2. The breathing simulation system according to claim 1, wherein the air suction mechanism includes:
a suction pump disposed at one end of the main tube; and
an air extraction controller electrically connected to the suction pump, wherein the air extraction controller is configured to turn on and off the suction pump with a predetermined pattern, such that the negative pressure changes in the main airflow passage according to the predetermined pattern.

3. The breathing simulation system according to claim 2, further comprising:
a barometer disposed in the main airflow passage, wherein the barometer is configured to detect a pressure in the main airflow passage,
wherein the air extraction controller adjusts the negative pressure generated in the main airflow passage according to the pressure detected by the barometer.

4. The breathing simulation system according to claim 1, further comprising a liquid supply mechanism that includes a plurality of liquid supply containers, wherein each of the liquid supply containers is used to accommodate a test liquid and is connected to the corresponding to-be-tested atomization device through a liquid supply tube, and the test liquid is provided to a liquid storage tank of the corresponding to-be-tested atomization device through the liquid supply tube.

5. The breathing simulation system according to claim 1, wherein the main tube is arranged along a direction of gravity and has an upper end and a lower end, the main tube is connected to a lower plate at the lower end, and the at least one liquid outlet is arranged on the lower plate.

6. The breathing simulation system according to claim 4, further comprising an aerosol condensation module that is arranged in the main tube, and is used to condense at least a part of aerosol generated during a test process performed by the to-be-tested atomization devices into a waste liquid, and discharge the waste liquid through the at least one liquid outlet.

7. The breathing simulation system according to claim 6, wherein the main tube has an upper end and a lower end, the air suction mechanism is configured to generate the negative pressure from the upper end, and the aerosol condensation module includes one or more baffles adjacent to the upper end, when the negative pressure is generated, at least a part of the aerosol is blocked by the one or more baffles to condense into the waste liquid on the one or more baffles, such that the waste liquid flows downward by an effect of gravity to be discharged from the at least one liquid outlet.

8. The breathing simulation system according to claim 4, further comprising:
a plurality of positioning mechanisms arranged on a periphery of the main tube, wherein each of the positioning mechanisms has an inclined surface inclined at a predetermined angle relative to ground, the inclined surface is used to carry the corresponding to-be-tested atomization device, such that the corresponding liquid storage tank is inclined relative to the ground at the predetermined angle.

9. The breathing simulation system according to claim 8, wherein each of the connecting pipes is inclined relative to the ground at the predetermined angle.

10. The breathing simulation system according to claim 1, wherein an airtight member is provided at a connection between each of the connecting pipes and the corresponding air suction port for sealing the connection.

11. The breathing simulation system according to claim 6, wherein the test process includes:
configuring the air suction mechanism to apply the negative pressure to each of the air suction ports through each of the airflow paths in a predetermined pattern; and
configuring each of the to-be-tested atomization devices to atomize the test liquid in the liquid storage tank when the negative pressure is detected by a first sensor to generate the aerosol to flow into the main airflow passage.

12. The breathing simulation system according to claim 11, wherein the liquid supply mechanism is configured to supply the corresponding test liquid into the corresponding liquid storage tanks according to consumptions of the liquid storage tanks, respectively.

13. The breathing simulation system according to claim 11, further comprising:
a breathing feature simulator electrically connected to the air suction mechanism, wherein the breathing feature simulator is configured to generate one or more of a plurality of breathing features when the negative pressure is generated, and the breathing features include a breathing sound, a breathing action, and one or more breathing signals generated by detecting respirational biological characteristics.

14. The breathing simulation system according to claim 13, wherein the test process further includes configuring each of the to-be-tested atomization devices to atomize the test liquid in the liquid storage tank when one or more of the breathing features are detected by a second sensor, so as to generate the aerosol to flow into the main airflow passage.

15. A breathing simulation system for testing a to-be-tested atomization device, and the breathing simulation system comprising:
a main tube forming a main airflow passage, wherein the main tube is provided with an air intake hole and at least one liquid outlet;
a connecting pipe disposed between the air intake hole and an air suction port of the to-be-tested atomization device; and
an air suction mechanism communicating with the main airflow passage, wherein the air suction mechanism is configured to generate a negative pressure in the main airflow passage, and form an airflow path between the air suction mechanism and the air suction port.

16. The breathing simulation system according to claim 15, further comprising a liquid supply mechanism that includes a plurality of liquid supply containers, wherein each of the liquid supply containers is used to accommodate a test liquid and is connected to the corresponding to-be-tested atomization device through a liquid supply tube, and the test liquid is provided to a liquid storage tank of the corresponding to-be-tested atomization device through the liquid supply tube.

17. The breathing simulation system according to claim 15, further comprising an aerosol condensation module that is arranged in the main tube, and is used to condense at least a part of aerosol generated during a test process performed by the to-be-tested atomization devices into a waste liquid, and discharge the waste liquid through the at least one liquid outlet.
